(19) United States
(12) Patent Application Publication
(10) Pub. No.: US 2019/0142838 A1
(43) Pub. Date: May 16, 2019

(54) METHODS FOR TREATING PARKINSON'S DISEASE

(71) Applicant: Reviva Pharmaceuticals, Inc., Sunnyvale, CA (US)

(72) Inventors: Laxminarayan BHAT, Cupertino, CA (US); Marc Cantillon, Livingston, NJ (US); Seema Rani Bhat, Cupertino, CA (US)

(21) Appl. No.: 16/247,407

(22) Filed: Jan. 14, 2019

Related U.S. Application Data
(63) Continuation-in-part of application No. 15/643,782, filed on Jul. 7, 2017, now abandoned.
Application No. 15/643,782 is a continuation of application No. PCT/US2016/013069, filed on Jan. 12, 2016.
(60) Provisional application No. 62/102,540, filed on Jan. 12, 2015.

(51) Int. Cl. A61K 31/538; A61P 25/16

(52) U.S. Cl. CPC A61K 31/538; A61P 25/16

(57) ABSTRACT

The present invention provides a method of using arylpiperazine derivatives for treating Parkinson's disease. The method is particularly effective for treating cognitive impairment associated with Parkinson's disease. The method comprises a step of administering to a patient in need thereof an effective amount of a compound of Formula I, which is an arylpiperazine derivative.

This application is a continuation-in-part of U.S. application Ser. No. 15/643,782, filed Jul. 7, 2017; which is a continuation of PCT/US2016/013069, filed Jan. 12, 2016; which claims the priority of U.S. Provisional Application No. 62/102,540, filed Jan. 12, 2015. The contents of the above-identified applications are incorporated herein by reference in their entireties.

### TECHNICAL FIELD

The present invention relates to methods of utilizing arylpiperazine derivatives for treating Parkinson's disease. The method is particularly effective for treating cognitive impairment associated with Parkinson's disease.

### BACKGROUND

Parkinson's disease (PD) is the second most important age-related neurodegenerative disorder globally, after Alzheimer's disease, with a prevalence ranging from 41 per 100,000 in the fourth decade of life to over 1900 per 100,000 in people over 80 years of age. The PD patients suffer from both motor and non-motor symptoms. The major motor symptoms are rigidity, resting tremor, and bradykinesia, and cognitive impairment is one of the most common non-motor symptoms besides psychosis, depression and dementia. Similar to motor symptoms, the characteristics of cognitive impairment in PD can be quite variable, both in terms of what cognitive domains are impaired and the timing of onset and rate of progression. Cognitive impairment in PD is characterized by predominant executive function deficits, attention difficulties, visuospatial dysfunction, slowed thinking, difficulties in word-finding, and difficulties in learning and remembering information.

Cognitive changes in patients can range from Parkinson's disease mild cognitive impairment (PD-MCI) to Parkinson's disease dementia (PDD). PD-MCI can be detected only by various means of comprehensive neuropsychological observations and normally does not affect the patients' daily operations. PDD hits more than one area of cognition and is severe enough to impair social or working functions. It can be difficult to differentiate PDD from Alzheimer's dementia, although Alzheimer's typically causes more pronounced memory loss and confusion, and also lacks the motor symptoms of Parkinson's (although stiffness and slowness may develop in very late stages). When dementia starts at the same time or within a year of the onset of Parkinson's motor symptoms, Lewy body dementia (LBD), a form of atypical parkinsonism, may be the cause. LBD is characterized by dementia and the motor symptoms of Parkinson's as well as fluctuating levels of alertness and visual hallucinations.

Conventional pharmacological treatments for PD are dopamine precursors (levodopa, 1-DOPA, 1-3,4 dihidroxifenilalanina), and other symptomatic treatments including dopamine agonists (amantadine, apomorphine, bromocriptine, cabergoline, lisuride, pergolide, pramipexole, ropinirole, rotigotine), monoamine oxidase (MAO) inhibitors (selegiline, rasagiline), and catechol-O-methyltransferase (COMT) inhibitors (entacapone, tolcapone). The chronic administration of antiparkinsonian drugs currently induces the “wearing-off phenomenon”, with additional psychomotor and autonomic complications. However, currently there is no approved medications for the treatment of PD-MCI and LBD. Exelon (rivastigmine), an inhibitor of acetylcholine, is the only FDA-approved for the treatment of mild to moderate levels of PDD but this treatment is far from optimal.

Thus, there is a need for more effective therapies for treating Parkinson's disease and cognitive impairment conditions (PD-MCI, PDD and LBD) associated with Parkinson's Diease.

### BRIEF DESCRIPTION OF THE DRAWINGS

FIGS. 1A-1C shows the effects of Compound A (RP5063) on novel object recognition (NOR) in male C57BL/6J mice. FIG. 1A (Acquisition Trial) shows that there was no preference for the left or the right object. FIG. 1B (Retention Trial) shows that Vehicle (Veh)+scVeh; and Veh+RP5063 (1.0 mg/kg, i.p.) had a significant increase in the exploration of novel object compared to familiar object (***p<0.001). Data are expressed as mean±SEM (N=10 per group). FIG. 1C (DI: Discrimination Index) shows that RP5063 (1.0 mg/kg, i.p.) significantly reversed the scPCP-induced decrease in DI (###p<0.001) and was not significantly different from the vehicle (veh)-treated group.

### DETAILED DESCRIPTION OF THE INVENTION

### Definitions

“Alkyl” or “alkanyl” refers to a saturated, branched or straight-chain or cyclic monovalent hydrocarbon radical derived by the removal of one hydrogen atom from a single carbon atom of a parent alkane. Typical alkyl groups include, but are not limited to methyl; ethyl; propyls such as propan-1-yl, propan-2yl, cyclopropan-1-yl; butyls such as butan-1-yl, butan-2-yl, 2-methyl-propan-1-yl, 2-methyl-propan-2-yl, cyclobutan-1-yl and the like. Preferably, an alkyl group comprises from 1-20 carbon atoms, more preferably, from 1 to 10, or 1 to 6, or 1-4 carbon atoms.

“Alkenyl” refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl;

propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methy-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien 1-yl, etc.; and the like.

“Alkynyl” refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn3-yl, but-3-yn-1-yl, etc.; and the like.

“Acyl” refers to a radical —C(O)R, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to formyl, acetyl, cyclohexylcarbonyl, cyclohexylmethylcarbonyl, benzoyl, benzylcarbonyl and the like.

“Acyloxyalkyloxycarbonyl” refers to a radical —C(O)OCR′R″OC(O)R′″, where R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —C(O)OCH₂OC(O)CH₃, —C(O)OCH₂OC(O)CH₂CH₃, —C(O)OCH(CH₃)OC(O)CH₂CH₃, —C(O)OCH(CH₃)OC(O)C₆H₅ and the like.

“Acylalkyloxycarbonyl” refers to a radical —C(O)OCR′R″C(O)R′″, where R′, R″, and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —C(O)OCH₂C(O)CH₃, —C(O)OCH₂C(O)CH₂CH₃, —C(O)OCH(CH₃)C(O)CH₂CH₃, —C(O)OCH(CH₃)C(O)C₆H₅ and the like.

“Acyloxyalkyloxycarbonylamino” refers to a radical —NRC(O)OCR′R″OC(O)R′″, where R, R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —NHC(O)OCH₂OC(O)CH₃, —NHC(O)OCH₂OC(O)CH₂CH₃, —NHC(O)OCH(CH₃)OC(O)CH₂CH₃, —NHC(O)OCH(CH₃)OC(O)C₆H₅ and the like.

“Acylalkyloxycarbonylamino” refers to a radical —NRC(O)OCR′R″C(O)R′″, where R, R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —NHC(O)OCH₂C(O)CH₃, —NHC(O)OCH₂C(O)CH₂CH₃, —NHC(O)OCH(CH₃)C(O)CH₂CH₃, —NHC(O)OCH(CH₃)C(O)C₆H₅ and the like.

“Acylamino” refers to “amide” as defined herein.

“Alkylamino” means a radical —NHR where R represents an alkyl, or cycloalkyl group as defined herein that may be optionally substituted by one or more substituents as defined herein. Representative examples include, but are not limited to, methylamino, ethylamino, 1-methylethylamino, cyclohexylamino and the like.

“Alkoxy” refers to a radical —OR where R represents an alkyl, or cycloalkyl group as defined herein that may be optionally substituted by one or more substituents as defined herein. Representative examples include, but are not limited to methoxy, ethoxy, propoxy, butoxy, cyclohexyloxy and the like.

“Alkoxycarbonyl” refers to a radical —C(O)-alkoxy where alkoxy is as defined herein.

“Alkoxycarbonylalkoxy” refers to a radical —OCR′R″C(O)-alkoxy where alkoxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OCH₃, —OCH₂C(O)OCH₂CH₃, —OCH(CH₃)C(O)OCH₂CH₃, —OCH(C₆H₅)C(O)OCH₂CH₃, —OCH(CH₂C₆H₅)C(O)OCH₂CH₃, —OC(CH₃)(CH₃)C(O)OCH₂CH₃, and the like.

“Alkoxycarbonylalkylamino” refers to a radical —NRCR′R″C(O)-alkoxy where alkoxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OCH₃, —N(CH₃)CH₂C(O)OCH₂CH₃, —NHCH(CH₃)C(O)OCH₂CH₃, —NHCH(C₆H₅)C(O)OCH₂CH₃, —NHCH(CH₂C₆H₅)C(O)OCH₂CH₃, —NHC(CH₃)(CH₃)C(O)OCH₂CH₃, and the like.

“Alkylsulfonyl” refers to a radical —S(O)₂R where R is an alkyl, or cycloalkyl group as defined herein that may be optionally substituted by one or more substituents as defined herein. Representative examples include, but are not limited to, methylsulfonyl, ethylsulfonyl, propylsulfonyl, butylsulfonyl, and the like.

“Alkylsulfinyl” refers to a radical —S(O)R where R is an alkyl, or cycloalkyl group as defined herein that may be optionally substituted by one or more substituents as defined herein. Representative examples include, but are not limited to, methylsulfinyl, ethylsulfinyl, propylsulfinyl, butylsulfinyl, and the like.

“Alkylthio” refers to a radical —SR where R is an alkyl or cycloalkyl group as defined/herein that may be optionally substituted by one or more substituents as defined herein. Representative examples include, but are not limited to methylthio, ethylthio, propylthio, butylthio, and the like.

“Amide” or “acylamino” refers to a radical —NR′C(O)R″, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, formylamino acetylamino, cyclohexylcarbonylamino, cyclohexylmethylcarbonyl-amino, benzoylamino, benzylcarbonylamino and the like.

“Aryl” refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorine, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleidene, pyrene, pyranthrene, rubicene, triphenylene, trinaphthalene, and the like. Preferable, an aryl group comprises from 6 to 20 carbon atoms, more preferably, between 6 to 12 carbon atoms.

“Arylalkyl” refers to an acyclic alkyl in which one of the hydrogen atoms bonded to a carbon atom, typically a terminal or sp³ carbon atom, is replaced with an aryl group. Typically arylalkyl groups include, but not limited to, benzyl, 2-phenylethan-1-yl, naphthylmethyl, 2-naphthylethan-1-yl, naphthobenzyl, 2-naphthophenylethan-1-yl and the like. Preferably, an arylalkyl group is (C₆-C₃₀)arylalkyl, e.g., the alkyl moiety of the arylalkyl group is (C₁-C₁₀) and the aryl moiety is (C₆-C₂₀), more preferably, an arylalkyl group is (C₆-C₂₀) arylalkyl, e.g., the alkyl moiety of the arylalkyl group is (C₁-C₈) and the aryl moiety is (C₆-C₁₂).

“Arylalkoxy” refers to an —O-arylalkyl radical where arylalkyl is as defined herein that may be optionally substituted by one or more substituents as defined herein.

“Arylalkoxycarbonylalkoxy” refers to a radical —OCR′R″C(O)-arylalkoxy where arylalkoxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OCH₂C₆H₅, —OCH(CH₃)C(O)O CH₂C₆H₅, —OCH(C₆H₅)C(O)O CH₂C₆H₅, —OCH(CH₂C₆H₅)C(O)O CH₂C₆H₅, —OC(CH₃)(CH₃)C(O)O CH₂C₆H₅, and the like.

“Arylalkoxycarbonylalkylamino” refers to a radical —NRCR′R″C(O)-arylalkoxy where arylalkoxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OCH₂C₆H₅, —N(CH₃)CH₂C(O)OCH₂C₆H₅, —NHCH(CH₃)C(O)OCH₂C₆H₅, —NHCH(C₆H₅)C(O)OCH₂C₆H₅, —NHCH(CH₂C₆H₅)C(O)OCH₂C₆H₅, —NHC(CH₃)(CH₃)C(O)OCH₂C₆H₅, and the like.

“Aryloxycarbonyl” refers to radical —C(O)-O-aryl where aryl is defined herein that may be optionally substituted by one or more substituents as defined herein.

“Aryloxycarbonylalkoxy” refers to a radical —OCR′R″C(O)-aryloxy where aryloxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OC₆H₅, —OCH(CH₃)C(O)OC₆H₅, —OCH(C₆H₅)C(O)OC₆H₅, —OCH(CH₂C₆H₅)C(O)OC₆H₅, —OC(CH₃)(CH₃)C(O)OC₆H₅, and the like.

“Aryloxycarbonylalkylamino” refers to a radical —NRCR′R″C(O)-aryloxy where aryloxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OC₆H₅, —N(CH₃)CH₂C(O)OC₆H₅, —NHCH(CH₃)C(O)OC₆H₅, —NHCH(C₆H₅)C(O)OC₆H₅, —NHCH(CH₂C₆H₅)C(O)OC₆H₅, —NHC(CH₃)(CH₃)C(O)OC₆H₅, and the like.

“Carbamoyl” refers to the radical —C(O)NRR where each R group is independently, hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Carbamate” refers to a radical —NR′C(O)OR″, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, methylcarbamate (—NHC(O)OCH₃), ethylcarbamate (—NHC(O)OCH₂CH₃), benzylcarbamate (—NHC(O)OCH₂C₆H₅), and the like.

“Carbonate” refers to a radical —OC(O)OR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, methyl carbonate (—C(O)OCH₃), cyclohexyl carbonate (—C(O)OC₆H₁₁), phenyl carbonate (—C(O)OC₆H₅), benzyl carbonate (—C(O)OCH₂C₆H₅), and the like.

“Cycloalkyl” refers to a substituted or unsubstituted cylic alkyl radical. Typical cycloalkyl groups include, but are not limited to, groups derived from cyclopropane, cyclobutane, cyclopentane, cyclohexane, and the like. In a preferred embodiment, the cycloalkyl group is (C₃-C₁₀) cycloalkyl, more preferably (C₃-C₇) cycloalkyl.

“Cycloheteroalkyl” refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, imidazolidine, morpholine, piperazine, piperidine, pyrazolidine, pyrrolidine, quinuclidine, and the like.

“Cycloheteroalkoxycarbonyl” refers to a radical —C(O)—OR where R is cycloheteroalkyl as defined herein that may be optionally substituted by one or more substituents as defined herein.

“Dialkylamino” means a radical —NRR′ where R and R′ independently represent an alkyl or cycloalkyl group as defined herein that may be optionally substituted by one or more substituents as defined herein. Representative examples include, but are not limited to dimethylamino, methylethylamino, di-(1-methylethyl)amino, (cyclohexyl)(methyl)amino, (cyclohexyl)(ethyl)amino, (cyclohexyl)(propyl)amino, and the like.

“Ester” refers to a radical —C(O)OR, where R is alkyl, substituted alkyl, cycloalkyl, substituted cycloalkyl, cycloheteroalkyl, substituted cycloheteroalkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl as defined herein that may be optionally substituted by one or more substituents as defined herein. Representative examples include, but are not limited to, methyl ester (—C(O)OCH₃), cyclohexyl ester (—C(O)OC₆H₁₁), phenyl ester (—C(O)OC₆H₅), benzyl ester (—C(O)OCH₂C₆H₅), and the like.

“Ether” refers to a radical —OR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Halogen” means fluoro, chloro, bromo, or iodo.

“Heteroaryl” refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Preferred heteroaryl groups are those derived from thiophene, pyrrole, benzothiophene, benzofuran, indole, pyridine, quinoline, imidazole, oxazole and pyrazine.

“Heteroaryloxycarbonyl” refers to a radical —C(O) OR where R is heteroaryl as defined that may be optionally substituted by one or more substituents as defined herein.

“Heteroarylalkyl” refers to an acyclic alkyl radical in which one of the hydrogen atoms bonded to a carbon atom, typically a terminal or sp3 carbon atom, is replaced with a heteroaryl group. Preferably, the heteroarylalkyl radical is a 6-30 carbon membered heteroarylalkyl, e.g., the alkyl moiety of the heteroarylalkyl is 1-10 membered and the heteroaryl moiety is a 5-20 membered heteroaryl, more preferably, a 6-20 membered heteroarylalkyl, e.g., the alkyl moiety of the heteroarylalkyl is 1-8 membered and the heteroaryl moiety is a 5-12 membered heteroaryl.

“Oxo” means the divalent radical ═O.

“Pharmaceutically acceptable” means approved or approvable by a regulatory agency of the Federal or state government or listed in the U.S. Pharmacopoeia or other generally recognized pharmacopoeia for use in animals, and more particularly in humans.

“Pharmaceutically acceptable salt” refers to a salt of a compound of the invention, which is pharmaceutically acceptable and possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tertiary butylacetic acid, laurylsulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“Phosphate” refers to a radical —OP(O)(OR′)(OR″), where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Phosphonate” refers to a radical —P(O)(OR′)(OR″), where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Racemate” refers to an equimolar mixture of enantiomers of a chiral molecule.

“Substituted” refers to a group in which one or more hydrogen atoms are each independently replaced with the same or different substituents(s). Typical substituents include, but are not limited to, —X, —R⁵⁴, —O⁻, ═O, —OR⁵⁴, —SR⁵⁴, —S, ═S, —NR⁵⁴R⁵⁵, ═NR⁵⁴, —CX₃, —CF₃, —CN, —OCN, —SCN, —NO, —NO₂, ═N₂, —N₃, —S(O)₂O⁻, —S(O)₂OH, —S(O)₂₀R⁵⁴, —OS(O)₂O³¹, —OS(O)₂R⁵⁴, —P(O)(O—)₂, —P(O)(OR¹⁴)(O³¹), —OP(O)(OR⁵⁴)(OR⁵⁵), —C(O)R⁵⁴, —C(S)R⁵⁴, —C(O)OR⁵⁴, —C(O)NR⁵⁴R⁵⁵, —C(O)O—, —C(S)OR⁵⁴, —NR⁵⁶C(O)NR⁵⁴R⁵⁵, —NR⁵⁶C(S)NR⁵⁴R⁵⁵, —NR⁵⁷C(NR⁵⁶)NR⁵⁴R⁵⁵, and —C(NR⁵⁶)NR⁵⁴R⁵⁵, where each X is independently a halogen; each R⁵⁴, R⁵⁵, R⁵⁶ and R⁵⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁸R⁵⁹, —C(O)R⁵⁸ or —S(O)₂R⁵⁸ or optionally R⁵⁸ and R⁵⁹ together with the atom to which they are both attached form a cycloheteroalkyl or substituted cycloheteroalkyl ring; and R⁵⁸ and R⁵⁹ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl.

“Sulfate” refers to a radical —OS(O)(O)OR, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Sulfonamide” refers to a radical —S(O)(O)NR′R″, where R′ and R″ are independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein or optionally R′ and R″ together with the atom to which they are both attached form a cycloheteroalkyl or substituted cycloheteroalkyl ring. Representative examples include but not limited to azetidinyl, pyrrolidinyl, piperidinyl, morpholinyl, 4-(NR′″)-piperazinyl or imidazolyl group wherein said group may be optionally substituted by one or more substituents as defined herein. R′″ hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Sulfonate” refers to a radical —S(O)(O)OR, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Thio” means the radical —SH.

“Thioether” refers to a radical —SR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Treating” or “Treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically (e.g., stabilization of a discernible symptom), physiologically, (e.g., stabilization of a physical parameter), or both.

“Therapeutically effective amount” means the amount of a compound that, when administered to a patient for treating a disease, is sufficient to effect such treatment for the disease. The “therapeutically effective amount” will vary depending on the compound, the disease and is severity and the age, weight, etc., of the patient to be treated, and can be determined by one of skill in the art without undue experimentation.

The present invention is directed to a method for treating Parkinson's disease and cognitive impairment conditions (PD-MCI, PDD and LBD) associated with Parkinson's Diease.

### Compounds Useful in the Invention

Compounds of Formula (I) are useful for the present invention:

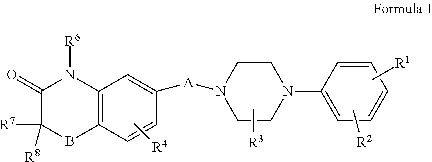

wherein:
A is —(CH₂)ₙ—, —O—(CH₂)ₙ—, —S—(CH₂)ₙ—, —S(O)(O)—(CH₂)ₙ—, —NH—(CH₂)ₙ—, —CH₂—O—(CH₂)ₙ—, —(CH₂)ₙ—O—CH₂—CH₂—, —CH₂—S—(CH₂)ₙ—, —(CH₂)ₙ—S—CH₂—CH₂—, —CH₂—S(O)(O)—(CH₂)ₙ—, —(CH₂)ₙ—S(O)(O)—CH₂—CH₂—, —O—C(O)—(CH₂)ₙ—, —S—C(O)—(CH₂)ₙ—, —NH—C(O)—(CH₂)ₙ—, —CH₂—C(O)—O—(CH₂)ₙ—, —CH₂—C(O)—NH—(CH₂)ₙ—, —CH₂—C(O)—S—(CH₂)ₙ—, —(CH₂)ₙ—C(O)—O—CH₂—CH₂—, —(CH₂)ₙ—C(O)—NH—CH₂—CH₂—, —(CH₂)ₙ—C(O)—S—CH₂—CH₂—, —CH₂—O—C(O)—(CH₂)ₙ—, —CH₂—NH—C(O)—(CH₂)ₙ—, —CH₂—S—C(O)—(CH₂)ₙ—, —(CH₂)ₙ—O—C(O)—CH₂—CH₂—, (CH₂)ₙ—NH—C(O)—CH₂—CH₂—, or (CH₂)ₙ—S—C(O)—CH₂—CH₂—, wherein n is an integer from 1 to 7, preferably n is 2 to 5, for example n is 4;
B is O, S, S(O)(O), or NR⁵; and
each of R¹, R², R³, R⁴, R⁵, R⁶, R⁷, and R⁸ is independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, acylalkyloxycarbonyl, acyloxyalkyloxycarbonyl, acylalkyloxycarbonylamino, acyloxyalkyloxycarbonylamino, alkoxy, alkoxycarbonyl, alkoxycarbonylalkoxy, alkoxycarbonyllalkylamino, alkylsulfinyl, alkylsulfonyl, alkylthio, amino, alkylamino, arylalkylamino, dialkylamino, arylalkoxy, arylalkoxycarbonylalkoxy, arylalkoxycarbonylalkylamino, aryloxycarbonyl, aryloxycarbonylalkoxy, aryloxycarbonylalkylamino, carboxy, carbamoyl, carbamate, carbonate, cyano, halo, heteroaryloxycarbonyl, hydroxy, phosphate, phosphonate, sulfate, sulfonate, or sulfonamide, wherein R¹, R², R³, R⁴, R⁵, R⁶, R⁷ and R⁸ and A may optionally be substituted with isotopes that include, but not limited to ²H (deuterium), ³H (tritium), ¹³C, ³⁶Cl, ¹⁸F, ¹⁵N, ¹⁷O, ¹⁸O, ³¹P, ³²P and ³⁵S; with ²H (deuterium) being preferred;
or a pharmaceutically acceptable salt, racemate or diastereomeric mixtures thereof.

In one aspect of the invention, A is —(CH₂)ₙ—.

In another aspect of the invention, A is —O—(CH₂)ₙ—, —CH₂—O—(CH₂)ₙ—, —(CH₂)ₙ—O—CH₂—CH₂—, —CH₂—S—(CH₂)ₙ—, or —(CH₂)ₙ—S—CH₂—CH₂—; with A being —O—(CH₂)ₙ— such as —O—(CH₂)₄— preferred.

In another aspect of the invention, A is —NH—C(O)—(CH₂)ₙ—, —CH₂—NH—C(O)—(CH₂)ₙ—, —CH₂—C(O)—NH—(CH₂)ₙ— or —(CH₂)ₙ—C(O)—NH—CH₂—CH₂—.

In another aspect of the invention, B is O.

In another aspect of the invention, R³, R⁴, R⁶, R⁶, and R⁸ are H.

In another aspect of the invention, each of R¹ and R² is independently H, halogen (e.g., chloro), haloalkyl, or alkoxy (e.g., methoxy or ethoxy); preferably halogen or alkoxy.

In a preferred embodiment, A is —O—(CH₂)ₙ—, n=2-5; B is O; R³, R⁴, R⁶, R⁶, and R⁸ are H; and R¹ and R² is independently H, halogen, haloalkyl, or alkoxy.

Preferred compounds of Formula I include, for example,

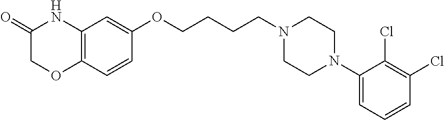 [SMILES: O=C1COc2ccc(OCCCCN3CCN(c4cccc(Cl)c4Cl)CC3)cc2N1]

6-(4-(4-(2,3-dichlorophenyl)piperazin-1-yl)butoxy)-2H-benzo[b][1,4]oxazin-3(-4H)-one, and its hydrochloride salt (Compound A); and

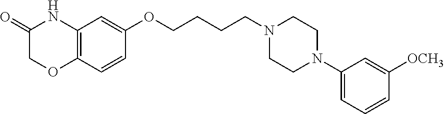 [SMILES: COc1cccc(N2CCN(CCCCOc3ccc4c(c3)NC(=O)CO4)CC2)c1]

6-(4-(4-(2-methoxyphenyl)piperazin-1-yl)butoxy)-2H-benzo[b][1,4]oxazin-3(-4H)-one, and its hydrochloride salt (Compound B).

The compounds useful for the present invention further pertain to enantiomerically isolated compounds of Formula I. The isolated enantiomeric forms of the compounds of Formula I are substantially free from one another (i.e., in enantiomeric excess). In other words, the “R” forms of the compounds are substantially free from the “S” forms of the compounds and are, thus, in enantiomeric excess of the “R” forms. Conversely, “S” forms of the compounds are substantially free of “R” forms of the compounds and are, thus, in enantiomeric excess of the “S” forms. In one embodiment of the invention, the isolated enantiomeric compounds are at least about in 80% enantiomeric excess. Thus, for example, the compounds are at least about 90% enantiomeric excess, preferably at least about 95% enantiomeric excess, more preferably at least about 97% enantiomeric excess, or even more preferably, at least 99% or greater than 99% enantiomeric excess.

Formula I compounds can be synthesized according U.S. Pat. No. 8,188,076, which is incorporated herewith in its entirety.

### Method of Treating Parkinson's Disease

The present invention is directed to a method for treating Parkinson's disease. The method comprises the step of administering an effective amount of a compound of Formula I to a patient in need thereof. Patients typically have a clinical diagnosis of Parkinson's Disease, defined as the presence of at least three of the following cardinal features, in the absence of alternative explanations or atypical features: rest tremor, rigidity, Bradykinesia and/or akinesia, and postural and gait abnormalities. Patients also have symptoms of cognitive impairment with at least one of the following features: Parkinson's disease mild cognitive impairment (PD-MCI), Parkinson's disease dementia (PDD) and Lewy body dementia (LBD).

The present invention is effective for treating cognitive impairment associated with Parkinson's disease. Pathobiology of Parkinson's disease involves imbalance of dopamine-serotonin systems in the brain (Dersi and Vecsei, Clin. Invest. 2011, 1:381-398). Compounds of Formula I have potent binding affinity at the serotonin 5-HT_{2A} receptor (compound A, Ki=2.5 nM, see Example 1) and 5-HT_{2B} receptor (compound A, Ki=0.19 nM, see Example 1). In addition, compounds of Formula I exhibit partial agonist activities for the key subtypes of dopamine (D₁, D₂, D₃ and D₄) and serotonin (5-HT_{1A}), and antagonist activity at the serotonin 5-HT6 and 5-HT₇ receptors. Compounds of Formula I are potent dopamine and serotonin system modulators due to their selectivity, potent binding affinities, and especially partial agonist activities for key dopamine and serotonin receptors. Due to the unique interaction of compounds of Formula I to various dopamine and serotonin receptors, the inventors have discovered that Formula I compounds are effective for treating cognitive impairment associated with Parkinson's disease.

In one embodiment, Formula I compounds treats cognitive impairment in patients having Parkinson's disease.

In one embodiment, Formula I compounds treats dementia in Parkinson's disease.

In one embodiment, Formula I compounds treats Lewy body dementia (LBD) in Parkinson's disease.

In one embodiment, Formula I compounds treats agitation in Parkinson's disease.

In one embodiment, Formula I compounds treats behavioral symptoms in Parkinson's disease.

In one embodiment, Formula I compounds treats depression in Parkinson's disease.

When used to treat patients with Parkinson's disease, one or more compounds of Formula I can be administered alone, or in combination with other agents, to a patient. The patient may be an animal, preferably a mammal, and more preferably a human.

Formula I compounds are preferably administered orally. Formula I compounds may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or composition of the invention. Methods of administration include, but are not limited to, intradermal, intramuscular, intraperitoneal, intravenous, subcutaneous, intranasal, epidural, oral, sublingual, intranasal, intracerebral, intravabinal, transdermal, rectally, by inhalation, or topically, particularly to the ears, nose, eyes or skin. Transdermal administration may be preferred for young children.

Formula I compounds can be delivered via sustained release systems, preferably oral sustained release systems. In one embodiment, a pump may be used (see, Langer, supra; Sefton, 1987, CRC Crit. Ref Biomed. Eng. 14:201; Saudek et al., 1989, N. Engl. J. Med. 321:574).

In one embodiment, polymeric materials can be used (see “Medical Applications of Controlled Release,” Langer and Wise (eds.), Wiley, New York (1984); Ranger and Peppas, 1983, J. Macromol. Sci. Rev. Macromol Chem. 23:61; see also Levy et al., 1985, Science 228:190; During et al., 1989, Ann. Neurol. 25:351; Howard et al, 1989, J. Neurosurg. 71:105). In a preferred embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropylmethylcellulose). Other preferred cellulose ethers have been described in the art (Bamba et al., Int. J. Pharm., 1979, 2, 307).

In one embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time controlled release), polymers that are degraded by enzymes (i.e., enzyme controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In a preferred embodiment, OROS® osmotic delivery systems are used for oral sustained release delivery devices (See for example, Theeuwes et al., U.S. Pat. No. 3,845,770; and Theeuwes et al, U.S. Pat. No. 3,916,899).

In yet another embodiment, a controlled-release system can be placed in proximity of the target of the compounds and/or composition of the invention, thus requiring only a fraction of the systemic dose (See, e.g., Goodson, in “Medical Applications of Controlled Release,” supra, vol. 2, pp. 115-138 (1984)). Other controlled-release systems discussed in Langer, 1990, Science 249:1527-1533 may also be used.

Formula I compounds may be cleaved either chemically and/or enzymatically. One or more enzymes present in the stomach, intestinal lumen, intestinal tissue, blood, liver, brain or any other suitable tissue of a mammal may enzymatically cleave the compounds and/or compositions of the invention.

### Pharmaceutical Formulation

The present invention is directed to a pharmaceutical formulation for treating patients with Parkinson's disease. The pharmaceutical formulation contains a therapeutically effective amount of one or more compounds of Formula I, preferably in purified form, together with a suitable amount of a pharmaceutically acceptable vehicle. When administered to a patient, the pharmaceutical formulation is preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present agents, or pH buffering agents. In addition, auxiliary, stabilizing, thickening, lubricating and coloring agents may be used.

Pharmaceutical compositions comprising a compound of the invention may be manufactured by means of conventional mixing, dissolving, granulating, levigating, and emulsifying, encapsulating, entrapping or lyophilizing process. Pharmaceutical compositions may be formulated in conventional manner using one or more physiologically acceptable carriers, diluents, excipients or auxiliaries, which facilitate processing of compounds of the invention into preparations which can be used pharmaceutically. Proper formulation is dependent upon the route of administration chosen.

The present compositions can take the form of solutions, suspensions, emulsion, tablets, pills, pellets, and capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 17^{th} Edition, 1985). Preferred compositions of the invention are formulated for oral delivery, particularly for oral sustained release administration.

Compositions for oral delivery may be in the form of tablets, lozenges, aqueous or oily suspensions, granules, powders, emulsions, capsules, syrups or elixirs, for example. Orally administered compositions may contain one or more optionally agents, for example, sweetening agents such as fructose, aspartame or saccharin; flavoring agents such as peppermint, oil of wintergreen, or cherry coloring agents and preserving agents to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about mM to about 50 mM) etc. Additionally, flavoring agents, preservatives, coloring agents, bile salts, acylcarnitines and the like may be added.

Compositions for administration via other routes may also be contemplated. For buccal administration, the compositions may take the form of tablets, lozenges, etc. formulated in conventional manner. Liquid drug formulations suitable for use with nebulizers and liquid spray devices and EHD aerosol devices will typically include a compound of the invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as alcohol, glycol, polyglycol or fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611). A compound of the invention may also be formulated in rectal or vaginal compositions such as suppositories or retention enemas, e.g., containing conventional suppository bases such as cocoa, butter or other glycerides. In addition to the formulations described previously, a compound of the invention may also be formulated as depot preparation. Such long acting formulations may be administered by implantation (for example, subcutaneously or intramuscularly) or by intramuscular injection. Thus, for example, a compound of the invention may be formulated with suitable polymeric or hydrophobic materials (for example, as an emulsion in an acceptable oil) or ion exchange resins, or as sparingly soluble derivatives, for example, as a sparingly soluble salt.

### Dosage for the Treatment

The amount of Formula I compound administered is dependent on, among other factors, the subject being treated, and the weight of the subject, the severity of the affliction, the manner of administration and the judgment of the prescribing physician. For example, the dosage may be delivered in a pharmaceutical composition by a single administration, by multiple applications or controlled release. In one embodiment, the compounds of the invention are delivered by oral sustained release administration. In one embodiment, the compounds of the invention are administered twice per day, and preferably, once per day. Dosing may be repeated intermittently, may be provided alone or in combination with other drugs, and may continue as long as required for effective treatment of the disease state or disorder.

The compounds of Formula I may be administered in the range 0.1 mg to 500 mg, preferably 1 mg to 100 mg per day, such as 5 mg, 10 mg, 15 mg, 20 mg, 25 mg, 35 mg or 50 mg per day, and preferably 10 mg per day.

### Combination Therapy

In certain embodiments of the present invention, the compounds of the invention can be used in combination therapy with at least one other therapeutic agent. Formula I compounds and the therapeutic agent can act additively or synergistically. In one embodiment, Formula I compound is administered concurrently with the administration of another therapeutic agent, which can be part of the same composition of Formula I compound. In another embodiment, a composition comprising a compound of the invention is administered prior or subsequent to administration of another therapeutic agent.

The invention is further illustrated by the following examples.

### Examples

### Example 1. In Vitro Pharmacology Results

Two arylpiperazine derivatives of Formula (I) were tested in the in vitro pharmacological assays to evaluate their activities for dopamine—D₁, D_{2L}, D_{2S}, D₃, D_{4.4}; serotonin-5-HT_{1A}, 5-HT_{2A}, 5-HT_{2B}, 5-HT6, 5-HT₇; serotonin transporter (SERT); and nicotinic acetylcholine alpha4beta2 (nACh-α₄β₂)serotonin. The radioligand binding assays were carried out at six to 10 different concentrations and the test concentrations were 0.1 nM, 0.3 nM, 1 nM, 10 nm, 30 nM, 100 nM, 300 nM, 1000 nM, 10000 nM. The in vitro assay protocols and literature references are described herein.

### Dopamine, D₁ Radioligand Binding Assay

### Materials and Methods:

Receptor Source: Human recombinant D₁ expressed CHO cells

### Radioligand: [3H]SCH 23390, 0.3 nM

### Control Compound: SCH23390

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 22° C. The reaction was terminated by rapid vacuum filtration onto glass fiber filters. Radioactivity trapped onto the filters was determined and compared to control values in order to ascertain any interactions of test compounds with the cloned dopamine—D₁ binding site.

### Dopamine, D_{2L} Radioligand Binding Assay

### Materials and Methods:

Receptor Source: Human recombinant D_{2L} expressed HET-293 cells

### Radioligand: [³H]Methylspiperone, 0.3 nM

### Control Compound: Butaclamol

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 22° C. The reaction was terminated by rapid vacuum filtration onto glass fiber filters. Radioactivity trapped onto the filters was determined and compared to control values in order to ascertain any interactions of test compounds with the cloned dopamine—D_{2L} binding site.

### Dopamine, D_{2S} Radioligand Binding Assay

### Materials and Methods:

Receptor Source: Human recombinant D_{2S} expressed CHO or HEK cells
Radioligand: [³H]Spiperone (20-60 Ci/mmol) or [3H]-7-hydroxy DPAT, 1.0 nM

### Control Compound: Haloperidol or Chlorpromazine

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25 C. The reaction was terminated by rapid vacuum filtration onto glass fiber filters. Radioactivity trapped onto the filters was determined and compared to control values in order to ascertain any interactions of test compounds with the cloned dopamine—D₂ short binding site (Literature Reference: Jarvis, K. R. et al. Journal of Receptor Research 1993, 13(1-4), 573-590; Gundlach, A. L. et al. Life Sciences 1984, 35, 1981-1988.)

### Dopamine, D_{4.4} Radioligand Binding Assay

### Materials and Methods:

Receptor Source: Human recombinant D_{2S} expressed CHO cells

### Radioligand: [³H]Spiperone, 0.3 nM, 1.0 nM

### Control Compound: (+)Butaclamol

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25 C. The reaction was terminated by rapid vacuum filtration onto glass fiber filters. Radioactivity trapped onto the filters was determined and compared to control values in order to ascertain any interactions of test compounds with the cloned dopamine—D_{4.4} binding site

### Dopamine, D₅ Radioligand Binding Assay

### Materials and Methods:

Receptor Source: Human recombinant D_{2S} expressed GH4 cells

### Radioligand: [3H]SCH 23390, 0.3 nM

### Control Compound: SCH 23390

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25° C. The reaction was terminated by rapid vacuum filtration onto glass fiber filters. Radioactivity trapped onto the filters was determined and compared to control values in order to ascertain any interactions of test compounds with the cloned dopamine—D₅ binding site

### Serotonin, 5HT_{1A} Radioligand Binding Assay

### Materials and Methods:

Receptor Source: Human recombinant 5-HT_{1A} expressed mammalian cells

### Radioligand: [³H]-8-OH-DPAT (221 Ci/mmol)

### Control Compound: 8-OH-DPAT

Incubation Conditions: The reactions were carried out in 50 mM TRIS-HCl (pH 7.4) containing 10 mM MgSO₄, 0.5 mM EDTA and 0.1% Ascorbic acid at room temperature for 1 hour. The reaction was terminated by rapid vacuum filtration onto glass fiber filters. Radioactivity trapped onto the filters was determined and compared to control values in order to ascertain any interactions of test compounds with the cloned serotonin 5HT_{1A} binding site (Literature Reference: Hoyer, D. et al. Eur. Journal Pharmacol. 1985, 118, 13-23; Schoeffter, P. and Hoyer, D. Naunyn-Schmiedeberg's Arch. Pharmac. 1989, 340, 135-138)

### Serotonin, 5HT_{2A} Radioligand Binding Assay

### Materials and Methods:

Receptor Source: Human Cortex or Human recombinant 5-HT_{2A} expressed mammalian cells

### Radioligand: [³H]-Ketanserin (60-90 Ci/mmol)

### Control Compound: Ketanserin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin-5HT_{2A} binding site (Literature Reference: Leysen, J. E. et al. Mol. Pharmacol. 1982, 21, 301-314; Martin, G. R. and Humphrey, P. P. A. Neuropharmacol. 1994, 33(3/4), 261-273.)

### Serotonin, 5HT_{2B} Radioligand Binding Assay

### Materials and Methods:

Receptor Source: Human recombinant 5-HT_{2B} expressed CHO-K1 cells
Radioligand: 1.20 nM [3H] Lysergic acid diethylamide (LSD)

### Control Compound: Ketanserin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin-5HT_{2B} binding site

### Serotonin, 5HT6 Radioligand Binding Assay

### Materials and Methods:

Receptor Source: Human recombinant 5-HT6 expressed mammalian cells

### Radioligand: [125I] SB258585, 15 nM or [³H]LSD, 2 nM

Control Compound: Methiothepin or serotonin
Incubation Conditions: The reactions were carried out in 50 mM TRIS-HCl (pH 7.4) containing 10 mM MgSO₄, 0.5 mM EDTA and 0.1% Ascorbic acid at room temperature for 1 hour. The reaction was terminated by rapid vacuum filtration onto glass fiber filters. Radioactivity trapped onto the filters was determined and compared to control values in order to ascertain any interactions of test compounds with the cloned serotonin-5HT6 binding site (Literature Reference: Gonzalo, R., et al., Br. J. Pharmacol., 2006 (148), 1133-1143)

### Serotonin, 5HT7 Radioligand Binding Assay

### Materials and Methods:

Receptor Source: Human recombinant 5-HT₇ expressed CHO cells
Radioligand: [3H] Lysergic acid diethylamide (LSD), 4 nM

### Control Compound: Serotonin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin-5HT₇ binding site
Nicotinic Acetylcholine α₄β₂ (nACh-α₄β₂) Radioligand Binding Assay

### Materials and Methods:

Receptor Source: Human recombinant nACh-α₄β₂ expressed mammalian cells

### Radioligand: [3H] Cytisine, 3.0 nM

### Control Compound: Epibatidine

Incubation Conditions: The reactions were carried out in 120 mM NaCl, 2.5 mM KCl, 50 mM Tris, 1 mM CaCl₂, 1 mM MgCl₂ containing buffer (pH 7.4) for 60 minutes at ambient temperature (37° C.). The reaction was terminated by rapid vacuum filtration onto glass fiber filters. Radioactivity trapped onto the filters was determined and compared to control values in order to ascertain any interactions of test compounds with the cloned nicotinic acetylcholine α₄β₂ (nACh-α₄β₂) binding site

### Serotonin Transporter (SERT) Radioligand Binding Assay

### Materials and Methods:

Receptor Source: Human recombinant H₁ expressed mammalian cells

### Radioligand: [3H] Citalopram, 2.0 nM

### Control Compound: Venlafaxine

Incubation Conditions: The reactions were carried out in 50 mM TRIS-HCl (pH 7.4) containing 120 mM NaCl, 5 mM KCl, 5 mM MgCl₂, 1 mM EDTA for 180 minutes at ambient temperature (37° C.). The reaction was terminated by rapid vacuum filtration onto glass fiber filters. Radioactivity trapped onto the filters was determined and compared to control values in order to ascertain any interactions of test compounds with the cloned serotonin transporter (SERT) site.

| Compound | Radioligand Binding Assay | Ki (nM) |
|---|---|---|
| A | Dopamine D1 | 100 |
| A | Dopamine D2L | 0.45 |
| A | Dopamine D2S | 0.28 |
| A | Dopamine D3 | 3.7 |
| A | Dopamine D4.4 | 6.0 |
| A | Serotonin 5-HT1A | 1.5 |
| A | Serotonin 5-HT2A | 2.5 |
| A | Serotonin 5-HT2B | 0.19 |
| A | Serotonin 5-HT6 | 51 |
| A | Serotonin 5-HT7 | 2.7 |
| A | Serotonin Transporter (SERT) | 107.1 |
| A | Nicotinic Acetylcholine α4β2 | 36.3 |
| B | Dopamine D2S | 0.30 |
| B | Serotonin 5-HT1A | 0.65 |
| B | Serotonin 5-HT2A | 111 |

### Example 2. Evaluation of the Effects of Compound A (RP5063) on Cognition Impairment and Memory in the Novel Object Recognition Task (NORT) Mouse Model

### Materials and Methods

Experiments were performed in 2.5-3 month old male C57BL/6J mice (Jackson, Mass., USA). Animals were group housed (five in a cage) in a controlled environment held at 21±2° and 50±15% relative humidity, with a 14:10 h light-dark period. All experiments were conducted during the light phase. Food and water were available ad libitum. All experiments were conducted in accord with the institutional Animal Care and Use Committee of Northwestern University, Chicago, Ill., USA. Pencyclidine (PCP) used for inducing cognitive impairment was supplied by the National Institute of Drug Abuse (Bethesda, Md., USA). Both PCP and RP5063 were dissolved or suspended in saline (Sal) solution (0.9% NaCl) and were administered intraperitoneally (i.p.), at a volume of 10 mL/kg body weight.

The experimental protocol for novel object recognition (NOR) testing in mice was adapted from the literature report (Hashimoto, et al, Eur. J. Pharmacol. 2005, 519:114-117). The NOR apparatus consisted of an open box made of Plexiglas (52 cM L; 52 cm W; 31 cm H) with white walls and a solid floor. The box was positioned approximately 30 cm above the floor centered on a table such that the overhead lights could not provide a spatial cue. Following the 7-day washout from subchronic drug or vehicle treatment on each of the three days prior to the acquisition trial, mice were habituated as a group to the empty NOR arena for an hour. The animals were habituated once after subcutaneous vehicle (scveh) or subcutaneous PCP (scPCP) injections prior to the first test, and were subsequently tested. During the acquisition trial, the animals were allowed to explore two identical objects (e.g. A1 and A2) for 10 minutes. This was followed by 24 hours inter-trial interval when the animals were returned to the home cage. During the retention trial, the animals were allowed to explore familiar object (e.g. A) from the acquisition trial and a novel object e.g. B). The location of the novel object in the retention trial was randomly assigned for each mouse tested using a pseudorandom schedule. The pseudorandom sequences followed the criteria suggested by Gellerman, 1933 and thus reduced the effects of object and place preference. Also to avoid bias or olfactory trails, we used objects in triplicates. Behavior was recorded on video for blind scoring of object exploration. The exploration time(s) of each object in each trial was recorded manually by the use of two stop watches. The discrimination index (DI) [time spent exploring the novel object−time spent exploring the familiar object)/total exploration time] was then calculated for retention trials. Mice were tested twice, with a gap of one week to 10 days between tests, to reduce carryover effects.

All data are expressed as the mean±S.E.M (N=10 per group). Exploration data (in the acquisition and retention trials) were analyzed by a two-way analysis of variance (ANOVA) followed by the pair-wise comparison when a significant effect was detected by the ANOVA. DI data was analyzed by one-way ANOVA followed by post hoc Bonferroni test when a significant effect was detected by ANOVA.

### Results

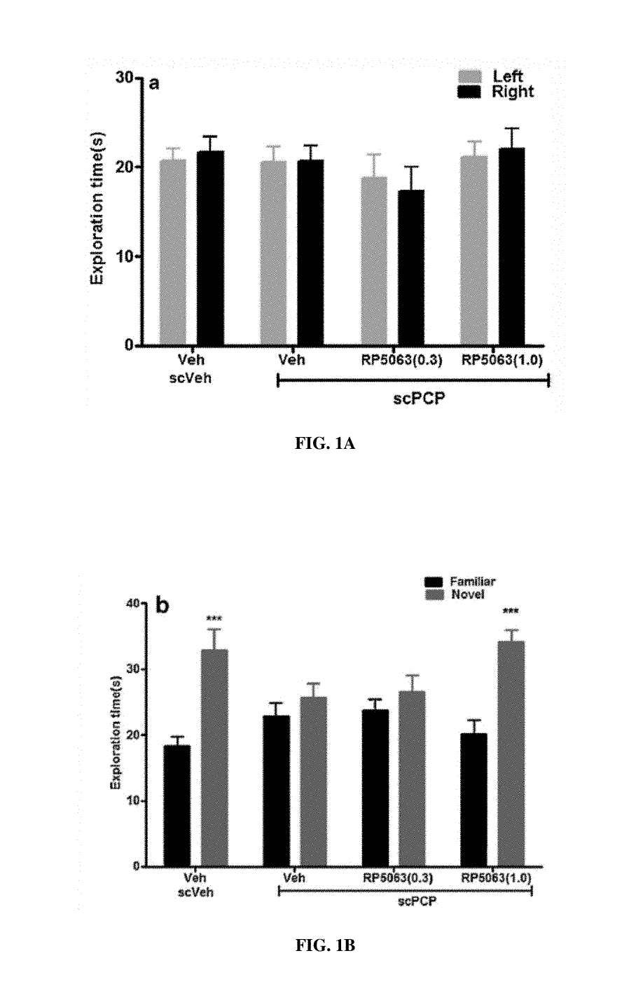

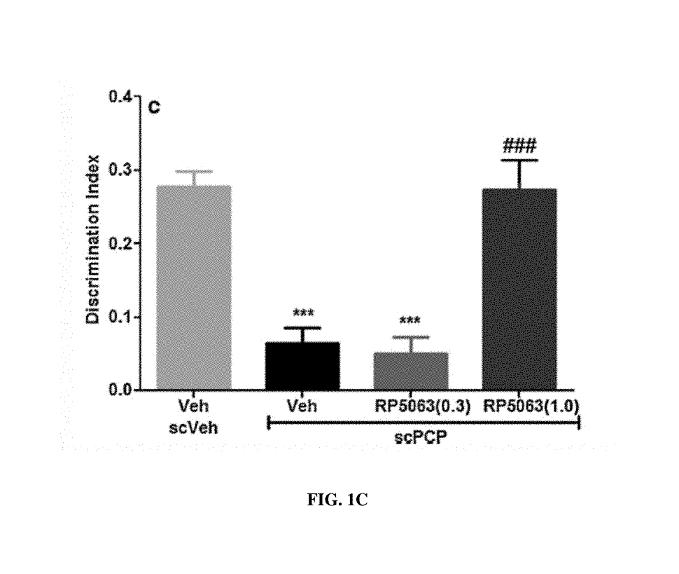

The ability of RP5063 to reverse the scPCP-induced deficit in NOR was tested. An overall two-way ANOVA of the acquisition trial showed no significant differences in the time spent exploring the two identical objects (p=0.63, FIG. 1A). Analysis with a two-way ANOVA of the exploration time for familiar and novel objects during the retention trial indicated a significant interaction of treatment group x object (***p<0.001). The veh group (***p<0.001), but not scPCP-treated group (p=0.97), showed significantly greater exploration of the novel vs the familiar object (FIG. 1B). Subchronic PCP (scPCP) treated mice given RP5063 (1.0 mg/kg, but not 0.3 mg/kg) reversed the scPCP-induced NOR deficit as indicated by significantly greater exploration of the novel vs the familiar object by the RP5063 (1 mg/kg) group (***p<0.001; FIG. 1B). A one-way ANOVA of the DI across treatment groups indicated statistically significant differences (***p<0.001; FIG. 1C). The DI of the scPCP-treated group, was significantly reduced compared to the veh group, scPCP induced an NOR deficit (***p<0.001; FIG. 1C). A single injection of RP5063 (1.0 mg/kg) significantly reversed the scPCP-induced decrease in DI (###p<0.001); and was not significantly different from the vehicle treated group (FIG. 1C).

In summary, Compound A (RP5063) is effective in reducing or reversing scPCP-induced memory deficit in mice

While the invention has been particularly shown and described with reference to a preferred embodiment and various alternate embodiments, it will be understood by persons skilled in the relevant art that various changes in form and details can be made therein without departing from the scope of the invention. All printed patents and publications referred to in this application are hereby incorporated herein in their entirety by this reference.

What is claimed is:

1. A method of treating Parkinson's disease, the method comprising administering to a patient in need thereof an effective amount of a compound of Formula 1: 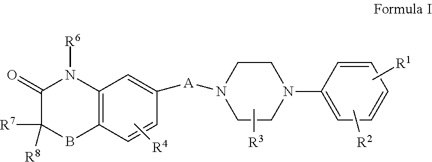
or a pharmaceutically acceptable salt, isomer, racemate, or diastereomeric mixture thereof, wherein:
A is —O—(CH₂)ₙ—, —(CH₂)ₙ—, —S—(CH₂)ₙ—, —CH₂—O—(CH₂)ₙ—, —(CH₂)ₙ—O—CH₂—CH₂—, —CH₂—S—(CH₂)ₙ—, —NH—C(O)—(CH₂)ₙ—, —CH₂—NH—C(O)—(CH₂)ₙ—, —CH₂—C(O)—NH—(CH₂)ₙ—, or —(CH₂)ₙ—C(O)—NH—CH₂—CH₂—, wherein n is an integer from 1 to 7;
B is O, S, S(O)(O), or NR⁵; and
R¹, R², R³, R⁴, R⁶, R⁷, and R⁸ are independently hydrogen, alkyl, substituted alkyl, aryl, substituted aryl, arylalkyl, substituted arylalkyl, cycloalkyl, substituted cycloalkyl, alkoxy, alkoxycarbonyl, alkylsulfinyl, alkylsulfonyl, alkylthio, amino, alkylamino, dialkylamino, arylalkoxy, carboxy, carbamoyl, carbamate, carbonate, cyano, halogen, or hydroxy; wherein the hydrogen of R¹, R², R³, R⁴, R⁶, R⁷ and R⁸ and A are optionally substituted with ²H (deuterium).

2. The method according to claim 1, wherein A is —O—(CH₂)ₙ—.

3. The method according to claim 1, wherein A is —(CH₂)ₙ—.

4. The method according to claim 1, wherein A is —NH—C(O)—(CH₂)ₙ—, —CH₂—NH—C(O)—(CH₂)ₙ—, —CH₂—C(O)—NH—(CH₂)ₙ—, or —(CH₂)ₙ—C(O)—NH—CH₂—CH₂—.

5. The method according to claim 1, wherein B is O.

6. The method according to claim 1, wherein R³, R⁴, R⁶, R⁷, and R⁸ are hydrogen.

7. The method according to claim 6, wherein R¹ and R² are independently H, halogen, or alkoxy.

8. The method according to claim 6, wherein R¹ is H, and R² is methoxy.

9. The method according to claim 6, wherein R¹ and R² are chloro.

10. The method according to claim 1, wherein A is —O—(CH₂)ₙ—; B is O, and R³, R⁴, R⁶, R⁷, and R⁸ are independently hydrogen or alkyl.

11. The method according to claim 1, wherein the compound is 6-(4-(4-(2,3-dichlorophenyl)piperazin-1-yl)butoxy)-2H-benzo[b][1,4]oxazin-3 (-4H)-one, or its hydrochloride salt thereof.

12. The method according to claim 1, wherein the compound is 6-(4-(4-(2-methoxyphenyl)piperazin-1-yl)butoxy)-2H-benzo[b][1,4]oxazin-3 (-4H)-one, or its hydrochloride salt thereof.

13. The method according to claim 1, wherein the compound is administered in a pharmaceutical composition comprising a pharmaceutically acceptable carrier, excipient, or diluent.

14. The method according to claim 1, wherein the compound is orally administered.

15. The method according to claim 1, which treats cognitive impairment in Parkinson's diease.

16. The method according to claim 1, which treats Parkinson's disease mild cognitive impairment (PD-MCI).

17. The method according to claim 1, which treats Parkinson's disease dementia (PDD).

18. The method according to claim 1, which treats Lewy body dementia (LBD).

19. The method according to claim 1, which treats agitation in the patient.

20. The method according to claim 1, which treats behavioral symptoms in Parkinson's patient.
